(19) United States
(12) Patent Application Publication
(10) Pub. No.: US 2010/0311003 A1
(43) Pub. Date: Dec. 9, 2010

(54) Oral respiration interface and a digital container

(76) Inventor: Aleksey Yuriy Kozlov, N. Highlands, CA (US)

Correspondence Address: Aleksey Yuriy Kozlov, 4109 Cabinet Cir., N. Highlands, CA, 95660, US

(21) Appl. No.: 12/802,383

(22) Filed: Jun. 4, 2010

Related U.S. Application Data
(60) Provisional application No. 61/217,908, filed on Jun. 4, 2009.

(51) Int. Cl. A61F 5/56; A61C 7/00

(52) U.S. Cl. 433/2; 128/848

(57) ABSTRACT

The invention relates to oral appliances configured to maintain users upper airway unobstructed, facilitating improved breathing and elimination of snoring and obstructive sleep apnea. This invention covers mechanical and pneumatic means of maintaining users airway open. A smart container for appliances is also disclosed to be used in conjunction with any oral appliance as a system for wirelessly recording patient biofeedback, treatment compliance and live monitoring of the users medical condition.

### CROSS-REFERENCE TO RELATED APPLICATION

this application claims priority under 35 U.S.C. §119(e) from U.S. provisional patent application No. 61/217,908 filed Jun. 4, 2009 the contents of which are incorporated herein by reference.

### BACKGROUND OF THE INVENTION

This invention pertains to oral appliances used to treat snoring and other disorders such as obstructive sleep apnea. Obstructive sleep apnea, is a cessation of breathing during one's sleep. This sleep disorder is treated by methods such as a surgery, oral appliance therapy, Positive Air Pressure (PAP) therapy or a combination involving several methods. PAP therapies are also employed to treat other medical and respiratory disorders, such as Cheynes-Stokes respiration, congestive heart failure, and stroke. A common PAP device comprises a flow generator (e.g., a blower) that delivers gas via delivery conduit to a patient interface. It is also known to deliver the PAP as a continuous positive airway pressure (CPAP), a variable airway pressure, such as bi-level pressure that varies with the patient's respiratory cycle or an auto-titrating pressure that varies with the monitored condition of the patient. Nasal, Oral-Nasal and Full Face masks are the common interfaces utilized for delivering PAP to the patient's airway. Unfortunately the use of these masks often exerts excessive pressure via head straps on the head and face often resulting in skin irritation, claustrophobia and eventually in therapy noncompliance. Also, severe cases of obstructive sleep apnea require uncomfortable PAP pressure settings to successfully treat the condition. Present invention provides an alternative solution to these predicaments.

### SUMMARY OF THE INVENTION

Oral appliance therapies utilizes a Mandibular Advancement Devices (MADs) and are becoming an increasingly acceptable alternative for patients who are nasal mask noncompliant. MAD appliances used in conjunction with PAP therapy have been found to treat severe sleep apnea more effectively while often requiring decreased air pressure setting. Patients having nasal obstruction due to anatomical conditions such as deviated septum are not able to benefit from nasal interfaces couple to MADs. Oral appliances are configured to mechanically open up the airway by translating the mandible forward. PAP therapy, on other hand, utilizes pneumatic pressure to splint the soft tissue in retroglossal area, therefore alleviating airway resistance which consequently prevents sleep apnea. Two therapies used together at once are more efficient.

The current invention is a device configured to introduce PAP therapy directly to the mouth without the need of head straps and a face mask. It can efficiently work in conjunction with MAD therapy. In case PAP pressure starts escaping patient's nose, plugs can be used or the PAP therapy conduit can be coupled to a splitter which is configured to deliver pressure to hollow nasal attachments and the oral device simultaneously. One skilled in the art can appreciate this solution provided by the present invention.

Negative pressure in the oral cavity has been taught to also open the obstructed airway by pulling the tongue forward and against the teeth and palate. This teaching can be satisfied with the present invention. The presented device can be configured with a vacuum generator to create a negative pressure in the oral cavity. When the device is configured to maintain subatmospheric pressure in the oral cavity, the patient can only breathe with the nose. To promote unrestricted nasal respiration, lateral pads can be added behind the lip to the right and left side of the appliance to lightly facilitate stretching of lateral nasal walls, therefore alleviating nasal airway resistance.

Further advancing the invention, the device can have a built in negative pressure generator. One approach to this is to implement an electronic negative pressure generator placed with a pressure sensor to maintain constant prescribed pressure. And another method is to have a manual membrane pump configured on the anterior perimeter of the oral device. In this case the user presses on the membrane by pressing on the cheeks, forcing the air out of the mouth. Pumping action is achieved utilizing at least two one-way valves positioned on anterior and posterior side of the membrane in relation to the dental arch. Positional memory of the compressible membrane forces it to regain original shape therefore sucking the air out of the oral cavity through the primary one-way valve which substantially keeps the air from leaking back into the oral cavity. The pneumatic mechanism that is behind the users lips naturally stretches the cheeks which in turn lightly dilates lateral nasal walls therefore promoting eased nasal respiration.

To monitor patients biofeedback an electronic system may be incorporated into the device and a device storage container, wherein the container will electromagnetically charge the device and transfer data from the device to and from the container and or the preferred network such as internet.

### BRIEF DESCRIPTION OF THE DRAWINGS

FIG. 1. illustrates an elevated side view of preferred embodiment wherein the lumen is attached to an oral appliance.

FIG. 2. illustrates a top (maxillary) view of preferred embodiment wherein the lumen is attached to an oral appliance.

FIG. 3. illustrates an elevated side view of preferred embodiment wherein the lumen is mechanically attached to an oral appliance employing a telescopic hinged mechanism fastened to maxillary and mandibular arch of an oral appliance.

FIG. 4. illustrates an elevated side view of hollow lumen detached besides an oral appliance.

FIG. 5a. illustrates a top view of preferred embodiment wherein the lumen is attached to an oral appliance. A segmented line is depicting a cross-section cut facing towards the exhaust port to help visualize FIG. 5b.

FIG. 5b. illustrates a cross-section view of the preferred embodiment, depicting lateral hollow air chambers created by the lumen positioned over maxillary and mandibular arch of the oral appliance.

FIG. 6. illustrates an elevated side view of a system in which the device disclosed herein is attached to an optional simultaneous nasal air-pressure delivery connected a conduit which is attached to the ventilator.

FIG. 7a. illustrates a top view of an oral appliance having an internal manual pump (in expanded state) for creating subatmospheric pressure in the oral cavity.

FIG. 7b. illustrates a top view of an oral device without the oral appliance attached, having an internal manual pump for creating subatmospheric pressure in the oral cavity, wherein one chamber is pressed by the user.

FIG. 8. illustrates a top view of an oral device with the hollow lumen attached to the oral appliance, employing an automatic internal electronic vacuum generator system.

FIG. 9. illustrates top view of an oral device employing the hollow lumen attached to an oral appliance having lateral nasal dilator pads attached to posterior aspect of the maxillary arch.

FIG. 10a. illustrates an elevated top view of closed electronic oral appliance container with a USB wire next to it.

FIG. 10b. illustrates a top view of opened electronic oral appliance container.

FIG. 11. illustrates a diagram of electronic systems embedded into the bottom of the electronic oral appliance container.

### DETAILED DESCRIPTION

In the following description, the use of “a,” “an,” or “the” can refer to the plural. All examples given are for clarification only, and are not intended to limit the scope of the invention.

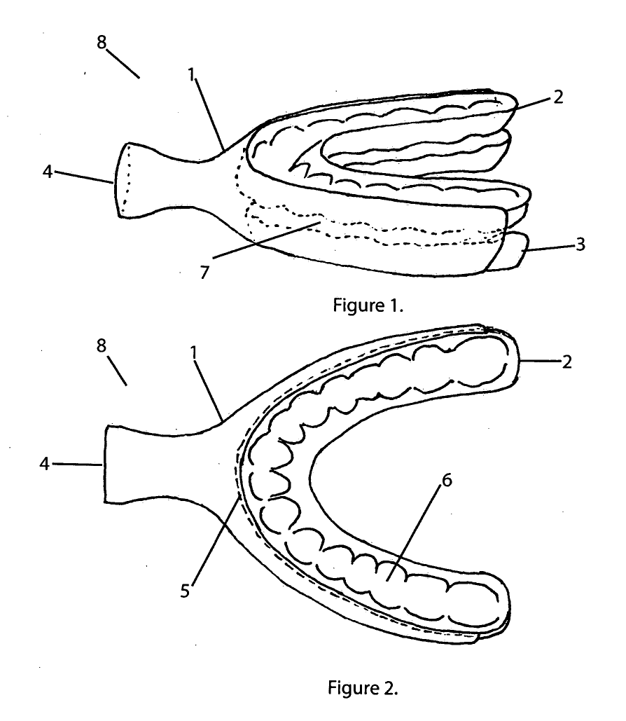

Referring to FIG. 1. illustrates an example device 8 for improving breathing which employs an oral appliance 2 and 3 having an upper arch 2 adapted to receive at least some of a user's maxillary teeth and a lower arch 3 adapted to receive at least some of the user's mandibular teeth wherein a hollow lumen 1 having an exhaust port 4 is attached to the anterior perimeter of the arches 2 and 3 wherein the arches have an occlusal gap that allows fluid gas communication between the oral cavity and an exhaust port 4. Arches 2 and 3 may be any arches suitable for dental or medical uses. For example, arches 2 and 3 may be custom arches that have been customized of otherwise formed, using a suitable deformable material, to fit the definition of a particular user. Suitable deformable materials may include, for example only and without limitation, methylmathacrylate, the polycarbonate resin thermoplasic such as that sold as BIOCRYL, the ethylene vinyl acetate copolymer resin sold under the name ELVAX, a thermoplastic polymer such as polycprolactone, or any other suitable deformable material. These materials are known, at least in certain contexts, to the skilled in the art, and other suitable materials may be used without departing from the intended scope of the invention.

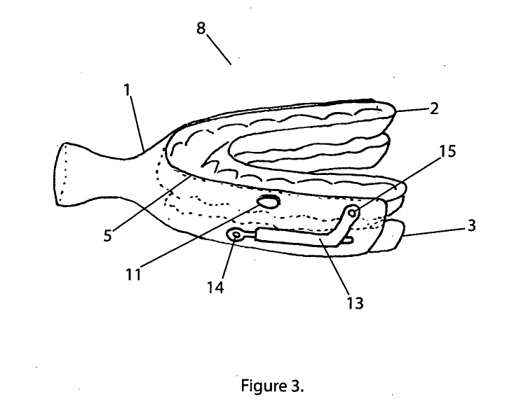

FIG. 2. shows a top view of the device 8 further illustrating the preferred embodiment. A bent collar 5 overlapping the maxillary arch 5 is shown. The collar 5 is used to stabilize the lumen 1 on the maxillary splint 2. Number 6 depicts the inside of an anatomic matrix of the teeth on the maxillary arch 2.

FIG. 3. illustrates an oral device 8 having the lumen 1 detachably connected to the maxillary arch 2 of the oral appliance 2 and 3 which is shown implementing a telescopic hinge mechanism 13, connected 15 to upper splint 2 having a telescoping shaft 14 connected to the lower splint. Because the lower splint 3 is not fused to the lumen 1, the user is able to open the mouth while maintaining prescribed mandibular forward position due to the hinge mechanism 14,13,15, while a positive or negative pressure is delivered to the users mouth through the hollow lumen 1.

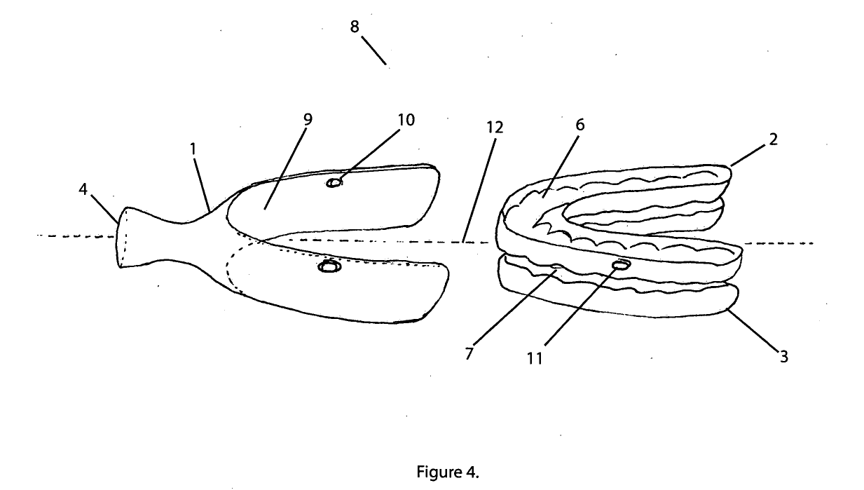

FIG. 4. further shows an insertion path for the lumen 1 and the oral disconnected appliance 2 and 3. It also illustrates a method for stabilizing the lumen 1 on the upper arch 2. lateral protrusions 11 are incorporated onto the upper splint 2 to mate with opposing concave matrixes 10 of the protrusions 11. When the protrusion 11 and the matrix 10 are locked, the lumen is immobile in relation to the upper splint 2.

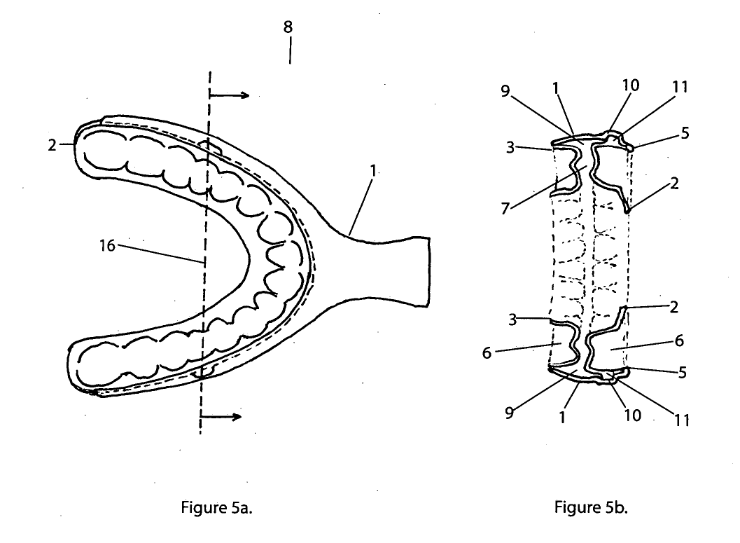

FIG. 5a. shows a cut line 16 in the device 8 to demonstrate the cross section on FIG. 5b.

FIG. 5b. is demonstrating an air canal 9 which delivers air anteriorly of the lower 3 and upper 2 arches all the way to the molar region of the splints while allowing the pressure to evenly communicate with the oral cavity through occlusal gap 7 distributed between the arches 2 and 3. FIG. 5b, further illustrates the locking action of the protrusions 11 with the matrix 10 incorporated into the lumen 1. Upper 2 and lower 3 splints snap over the teeth with a negative impression shaped splints 6.

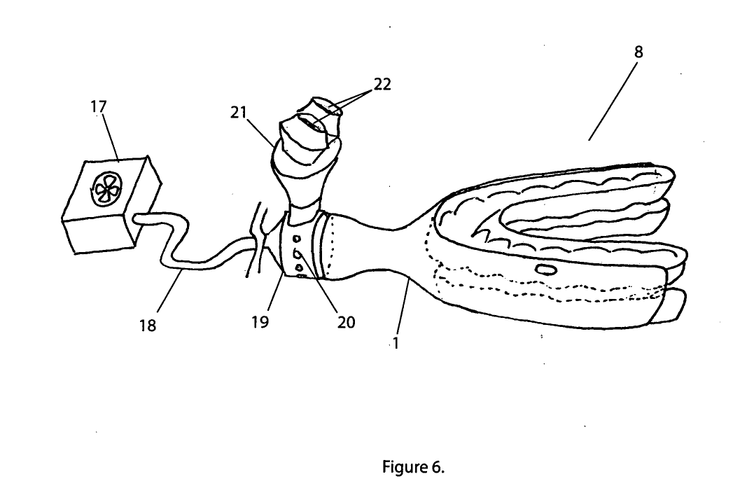

FIG. 6. illustrates a system in which the oral device 8 employing a hollow lumen 1 is connected to the ventilator 17 via a conduit 18 capable of delivering negative or positive pressure or even anesthesia. An optional nasal attachment 19 having a hollow body 21 is connected between the lumen 1 and a conduit or a ventilator (in an instance where positive pressure is used) having a nasal attachment ('s) 22 to be connected to the nose and ventilation holes 20 to let out exhaled air.

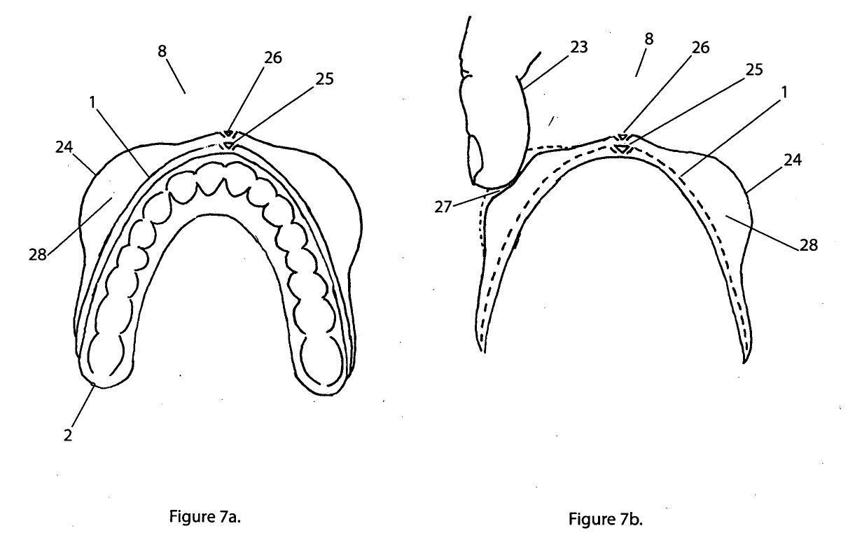

FIG. 7a. Illustrates a top view of an oral device 8 employing a built-in manual pump wherein the lumen 1 has no external exhaust port. The lumen 1 is like in previous embodiments, positioned over the arches 2 and 3. The lumen 1 has at least one one-way valve 25 allowing air to escape the oral cavity into the hollow space 28 wherein the valve closes when air is trying to re-enter the oral cavity. A flexible membrane 24 is positioned over the lumen 1 having a sealed around the perimeter of lumen 1 comprising a hollow space 28. The membrane 24 has at least one secondary one-way valve 26, allowing the air to escape out of the hollow space 28 through it 26 and into the atmosphere away from the user. This action creates subatmospheric pressure in the oral cavity, which in turn pulls the tongue forward sealing it between the splints 2 and 3, which in turn opens up the airway in the back of the throat, easing the breathing effort for the user. The bulging of the membranes 24, stretches the lip and cheek tissue, pulling on the lateral walls of the nasal airway, therefore easing nose breathing.

FIG. 7b. further illustrates a device 8 described in FIG. 7a. wherein it is not connected to any oral appliance. This as a stand-alone configuration which configured to be placed into the users mouth by it's self. It is preferred that the lumen 1 be able to conform to the anatomy of the user utilizing flexible surface or a boil-and-bite type of material. As in FIG. 7a, the members 24 are pressable 27 wherein the user can press with a finger 23 on the surface of the lateral membranes 24 while placed in the mouth to create suction. When enough suction is created, the membranes are slightly caved in, continuously maintaining negative pressure while trying to regain its previous expanded shape.

FIG. 8. illustrates a top view of an oral device 8 wherein the lumen 1 houses in its hollow cavity between the anterior region of the upper 2 and lower 3 oral appliance arches an automatic electronic negative pressure generator. The embedded system may comprise of a re-chargeable battery 32, connected to electronic components with a wire 38. The built in system may further comprise a (CPU) Central Processing Unit 31 that may contain various sensors such as an SPO2 sensor to measure the users pulse, oxygen saturation in blood and temperature sensor. Patient's compliance or biofeedback data may be transferred to out of the device via an antennae 33. The CPU 31 controls the wireless recharging of the battery 32 utilizing a coil 29 to absorb electromagnetic energy exerted by an external source. The CPU 31 is connected to a pressure sensor 35 which is used to maintain stable pre-programmed pressure in the oral cavity via pump 34. The pump 34 has an inlet 36 for air in the oral cavity and an outlet 30 that dispenses air out into the atmosphere away from users lips. This automatic device 8 may be constructed with the mandible set forward to further open up the airway in the retroglossal region, therefore furthering the efficacy of the device.

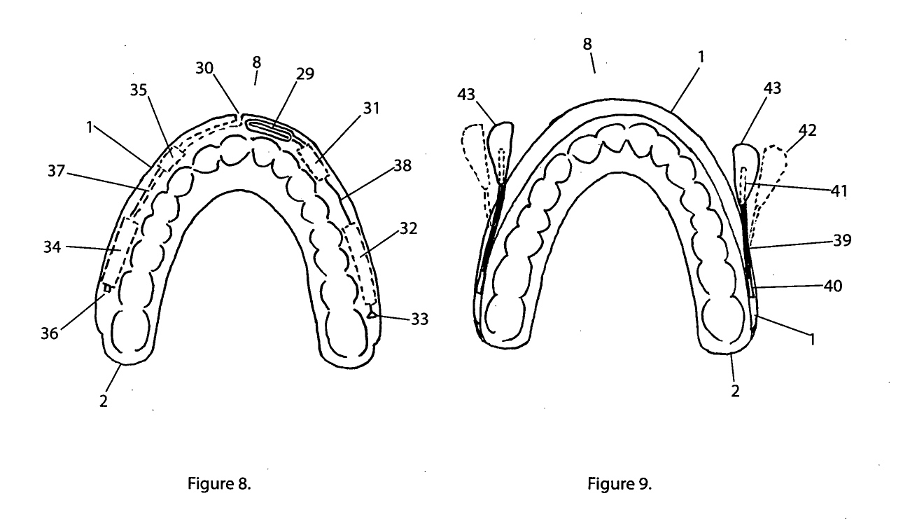

FIG. 9. illustrates an oral device 8 having a hollow lumen 1 without an exhaust port (can be constructed with exhaust port 4) detachably connected to the upper 2 and lower 3 splints wherein the oral device incorporates lateral tissue dilators 43. The lip and cheek tissue dilators 43 are pads connected with a flexible material 39 such as cobalt-chrome wire (but not limited to it) to the posterior 40 of the lumen 1 or maxillary splint 2. The pads can flex outward or inward 42 to accommodate patient's dilating force requirement. The stretched tissues enable lateral nasal walls to stretch open, allowing the user to breathe easier through the nose. Mandibular advancement via the oral appliance 2 and 3 further opens up the users airway. This embodiment can be configured to be a mandibular advancement device by bonding the lumen 1 to the lower splint 3 and having the lumen push against the anterior portion of the maxillary arch 2. This is an anterior bumper effect that prevents the mandible from going backwards yet allowing to move the jaw forward since the upper splint 2 is not bonded to the lumen 1.

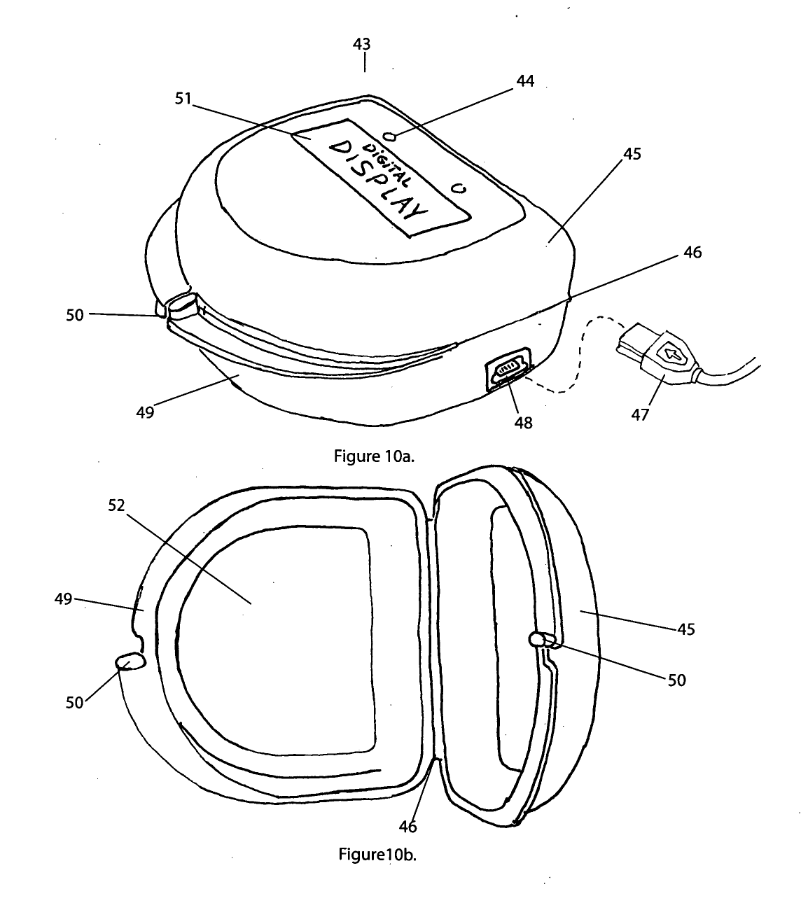

FIG. 10a. illustrates an elevated side view of the (wireless communication/charging station/oral appliance container) 43. Wherein the container 43 has a top lid containing a touch-screen digital display 51 a ventilation ports, a base 49 containing electronic components in the walls or the bottom, a USB 48 or other computer interface via a wire 47, a hinge 46 holding the two parts of the container and a locking mechanism 50. This “smart” container can be used with the device depicted in FIG. 8. or with any other medical or dental device. When the user of a medical/dental device is not using it, then he or she stores the device inside of the container where it can recharge and communicate with the device, the internet or other wireless or wired connection.

FIG. 10b. Illustrates the container in the open state. It is configured to contain the device inside. In this embodiment, most of the electronic components are located behind the bottom interior surface 52 of the container 43.

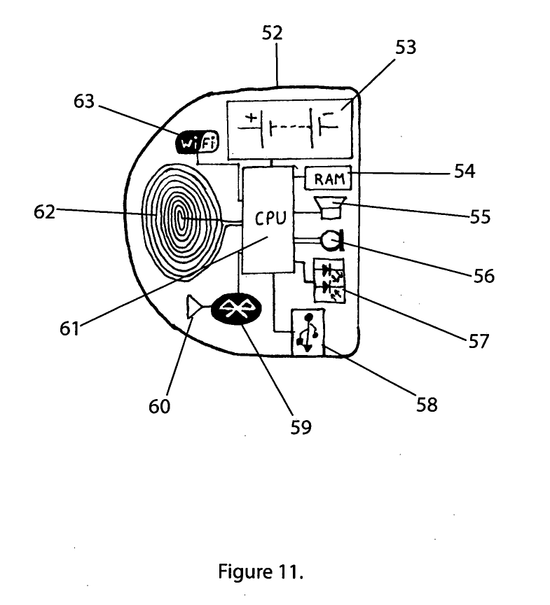

FIG. 11 Further illustrates the contents of the (wireless communication/charging station/oral appliance container) 43 that are positioned on the bottom interior surface 52. The electronic components comprise of a rechargeable battery 53, a Central Processing Unit (CPU) 61, Solid state memory RAM 54, a USB port 58, a BlueTooth 59 or equivalent system, a radio antennae 60 to communicate with the appliance and internet network, a WiFi 63 to establish communication with the world wide web network where the doctors can access patients data anytime, an electromagnetic coil 62 to create a magnetic field for wirelessly charging the medical/dental appliance, a Infra Red (IR) 57 communication port to support other devices on the market, a built in speaker 55 to allow patients hear prescribed commands regarding their treatment compliance or to communicate with the doctor or medical personnel by incorporating a microphone 56. The USB 58 port allows to preset custom programs on a Personal Computer which will satisfy different application needs depending what type of device is stored inside of the container 43. The user can use the display 51 to view reports and graphs of his or her pulse rates, oxygen level, compliance and other preconfigured data.

One useful application of such container is to keep it in proximity of wireless connection to the device when the patient is sleeping. In a case that the users heart fails or oxygen reached dangerous low levels, the oral device can send a signal to the “Smart” container which will to one or combination of the following (flash alarm lights, awaken the users spouse via sound, notify emergency personnel or send a signal back to the oral device to activate any pre set function such as a medicine injection into patients blood stream.

I claim:

1. A device for improving users breathing, comprising:
An oral appliance comprising an arch adapted to receive at least some of the users teeth to secure the oral appliance in position in the user's mouth; and
a hollow lumen having an anterior opening protruding outward between lips and detachably (or permanently coupled to the arch (or arches) of the oral appliance, the lumen having a perimeter adapted to be placed behind the user's lips and cheeks such that the lumen is operable to allow gas fluid to pass in and out of the oral cavity passing anteriorly and between maxillary and mandibular arches of the appliance.

2. A device of claim 1, configured to facilitate gas exchange between entire maxillary and a mandibular dental arches;
at least one lumen positioned anterior of right and/or on left side of at least one dental arch region, wherein gas is able to travel from said exchange opening to molar region of the device and then into the oral cavity through occlusal opening between teeth.

3. A device of claim 1, configured to facilitate gas exchange between maxillary and a mandibular arch of the oral appliance;
at least one lumen positioned anterior of right and/or on left side of at least one dental arch region, wherein gas is able to travel from said exchange opening to molar region of the device and then into the oral cavity through occlusal opening between maxillary and mandibular splint of oral appliance.

4. A device of claim 1, wherein an external positive air pressure source coupled to the anterior opening of the device for applying positive pressure to the user's airway.

5. A device of claim 1, wherein an anesthesia is delivered to the user through anterior opening.

6. A device of claim 1, wherein a negative air pressure generator is coupled to the anterior opening of the device for applying negative pressure to the user's airway.

7. A device of claim 6, configured to promote nasal breathing wherein the maxillary oral appliance comprises lateral extensions positioned between the maxilla and the upper lip such that a pressure applied to the upper lip expands the nasal channel.

8. A device of claim 3, where in the hollow lumen is attached to the maxilary arch of the oral appliance while the lower portion of the lumen is conformed yet not bonded to the mandibular arch of the appliance, facilitating jaw movement.

9. A device of claim 1, wherein the lumen is mechanically attached to the oral appliance comprising a ridge conforming to the edge of the maxillary or mandibular part of the oral device and lateral positive or negative protrusions corresponding to lateral positive or negative protrusions of the maxillary or mandibular part of the oral device configured to detachably mate the lumen to the oral appliance

10. A device of claim 7, wherein the portion of the lumen that conforms to the mandibular arch of the appliance is elastic.

11. A device of claim 1, wherein the lumen is configured to connect to the mandibular part of oral appliance and have the upper portion of the lumen conform to the anterior anatomy of dental or oral appliance arch configured to urge the lower jaw in an anterior direction relative to the maxillary arch when the connected lumen and mandibular oral appliance is mounted on the lower jaw.

12. An automatic electronic negative pressure generator inside a hollow lumen having coupled to the arch of the oral appliance, the lumen having a perimeter adapted to be placed behind the user's lips such that the lumen is operable to allow gas fluid to pass out of the oral cavity between maxillary and mandibular arches comprising;
a battery, a wire coil to receive external electromagnetic radiation for charging the battery, a Central Processing Unit for controlling the pressure in the oral cavity via pump, a pulse oximeter to measure users pulse rate and oxygen in blood, Infra Red communication port and a radio antennae to communicate with external data processing system.

13. A device of claim 10, comprising Pulse Oximeter sensors and pressure sensors employed to monitor patient's pulse rate, oxygen level in blood and atmospheric pressure within the oral cavity.

14. A container for storing the medical/dental electronic integrated device comprising; an electromagnetic coil for wirelessly charging the contained device, a wireless communication port to the internet and the device.

15. A container of claim 14 which comprises a digital touch-screen display, a sound generator, a microphone, a Bluetooth interface.

16. A system employing positive or negative pressure generator coupled via conduit to a device of claim 1

17. A device of claim 12, wherein the oral appliance comprises a mandibular arch coupled to the maxillary arch via hollow lumen in a fixed forward position.

18. A device of claim 12, configured to threat malocclusion of the teeth or jaw relation.

19. A device of claim 12, configured to splint and stabilize the jaw for post operation or post trauma healing process of the skeletal hard and soft tissues.

20. A device of claim 1, wherein the lumen has no extension outward between the lips and comprises a one-way valve configured to let air out of the oral cavity and into a pressable membrane which is sealed over perimeter of the lumen also containing a one way air valve configured to create negative pressure in the oral cavity and to stretch the lips which dilate nasal passages, promoting ease of nasal breathing.
